(19) United States
(12) Patent Application Publication
(10) Pub. No.: US 2004/0018151 A1
(43) Pub. Date: Jan. 29, 2004

(54) Use of anandamide/cannabinoid receptor/acceptor agonists in als screening and treatment methods

(76) Inventor: Mary E. Abood, Redwood City, CA (US)

Correspondence Address: Mary E. Abood, Forbes Norris ALS/MDA Research Center, Suite 416, 2351 Clay St., San Francisco, CA, 94115, US

(21) Appl. No.: 10/624,199

(22) Filed: Jul. 21, 2003

Related U.S. Application Data
(60) Provisional application No. 60/398,873, filed on Jul. 26, 2002.

(51) Int. Cl. A61K 49/00; A61K 31/353; A61K 31/16

(52) U.S. Cl. 424/9.1; 514/454; 514/627

(57) ABSTRACT

In one aspect of the present invention, a method of screening for compounds useful for promoting normal motor function in ALS patients is provided. The screening method comprises (a) administering a compound that is a anandamide/cannabinoid receptor/acceptor agonist to a mammal having observable motor function, and (b) evaluating one or more indicia of motor function in said mammal, wherein a compound that promotes normal motor function is identified. More preferably, the mammal to which the administration is made has one or more ALS or MND symptoms and such one or more symptoms includes at least one of the observable motor functions being evaluated.

### CROSS-REFERENCE TO RELATED APPLICATIONS

This application claims the benefit of provisional application Serial No. 60/398,873, filed Jul. 26, 2002, entitled “Use of THC to Extend the Survival of ALS Patients”, of common inventorship with this application.

### BACKGROUND OF THE INVENTION

2. Field of the Invention

This invention generally relates to screening for compounds useful in treatment and the treatment of ALS symptoms, and more particularly to the use of an anandamide/cannabinoid receptor/acceptor agonist to treat ALS-related symptoms in mammals, including rodents such as mice and rats, and primates such as chimpanzees and humans.

2. Description of Related Art

ALS and MND are abbreviations for Amyotrophic Lateral Sclerosis and Motor Neurone Disease respectively. ALS/MND is a neurological disease that affects over 350,000 of the world's population, and kills over 100,000 every year. Early symptoms associated with ALS include tripping, dropping things, slurred or “thick” speech, and muscle cramping, stiffening, weakening, and twitching (fasciculation). In Bulbar ALS, the muscles for speaking, swallowing or breathing are affected. In addition to muscle loss (atrophy/, the following signs of lower and upper motor neuron degeneration are often associated with ALS:

Lower Motor Neuron Degeneration

muscle weakness and atrophy

involuntary contraction of muscle fibres

muscle cramps

weakened reflexes

flaccidity (decreased muscle tone)

difficulty swallowing

disordered articulation

shortness of breath at rest

Upper Motor Neuron Degeneration

muscle stiffness or rigidity

emotional lability (decreased ability to control emotions).

Significant advances are being made towards understanding the genetic basis for ALS as well as the mechanistic and molecular pathways mediating progression of the sporadic forms of the disease, however, effective pharmacotherapy remains elusive. Two of the primary theories underlying motor neuron vulnerability are susceptibility to excitotoxicity and oxidative damage.

Recently published findings showed that delta⁹-THC and activation of the CB₁ cannabinoid receptor effectively reduce kainate toxicity in primary neuronal cultures prepared from mouse spinal cord (Abood et al, Neurosci Lett 309:197-201, 2001). Dronabinol (Marinol®) is a controlled substance that contains delta-9-tetrahydrocannabinol (THC), a major active ingredient found in marijuana. Dronabinol is approved for appetite stimulation in AIDS-related anorexia and treatment of chemotherapy-induced nausea and vomiting in patients who have failed to respond to conventional anti-emetic therapies. Dronabinol is sold in 2.5 mg, 5 mg and 10 mg capsules under the brand name Marinol. More recently, Dronabinol has been reported to be an effective treatment for the spasticity seen in patients with multiple sclerosis. However, the precise macromolecular system with which this drug is interacting is unknown.

### BRIEF SUMMARY OF THE INVENTION

Compounds that affect the anandamide/CB receptor system or any other macromolecular systems are believed to have the potential to reduce both excitotoxic and oxidative cell damage. This potentially synergistic action is believed useful in practicing the inventive method for a therapeutic effect, as compared to conventional glutamate antagonists or antioxidants.

In one aspect of the present invention, administering a predetermined amount of an anandamide/cannabinoid receptor/acceptor agonist, preferably selected from one or more of the cannabinoids, more preferably THC, a THC analog or an anandamide analog, to an ALS patient is used to protect motor neurons against excitotoxicity and oxidative damage and stress. Moreover, practice of the inventive therapeutic method can be used to slow the disease progression, reduce mortality, extend the survival and improve the quality of life in ALS patients.

A particularly preferred treatment in accordance with the invention is orally administering delta 9-THC (dronabinol).

In another aspect of this invention a screening method, useful to identify compounds affecting motor function in ALS or MND patients, comprises: administering an anandamide/cannabinoid receptor/acceptor agonist to a mammal having at least one detectable motor function related to an ALS or MND symptom; detecting any motor function change; and, evaluating any such motor function change with respect to a determinable motor function.

In yet another aspect of the present invention, a method of screening for compounds useful for promoting normal motor function in ALS patients is provided. The screening method involves (a) administering a compound that is a anandamide/cannabinoid receptor/acceptor agonist to a mammal having observable motor function, and (b) evaluating one or more indicia of motor function in said mammal, wherein a compound that promotes normal motor function is identified. More preferably, the mammal to which the administration is made has one or more ALS or MND symptoms and such one or more symptoms includes at least one of the observable motor functions being evaluated. Alternatively, the method can be used to identify a compound for promoting increased survival of ALS patients. In addition, indicia of excitotoxicity and oxidative damage can be used to evaluate the effects of administering the compound.

Other advantages and aspects of the present invention will be understood by reading the following detailed description and the accompanying claims.

### BRIEF DESCRIPTION OF THE DRAWINGS

FIGS. 1a and 1b graphically illustrate the delay of disease in hSOD1 mice treated in accordance with the invention;

FIG. 2 (parts a, b and c) graphically illustrates survival of treated mice; and,

FIG. 3 graphically illustrates attenuation of oxidative stress in treated mouse spinal cord cultures with respect to control.

### DETAILED DESCRIPTION OF THE INVENTION

The inventor has discovered that (a) anandamide/cannabinoid receptor is present in areas of the brain associated with motor function; (b) anandamide/cannabinoid receptor/acceptor agonists reduce excitotoxin-induced death of spinal cord cultures; (c) modulating anandamide/cannabinoid receptor in mammals by administering Dronabinol (delta-9-THC), an anandamide/cannabinoid receptor agonist, results in prolonged survival, and (d) administering delta-9-THC to human ALS patients results in improved symptoms, including improved motor function.

More particularly, administering a predetermined amount of an anandamide/cannabinoid receptor/acceptor agonist, preferably selected from one or more of the cannabinoids, more preferably THC, a THC analog or an anandamide analog, to an ALS patient is used to protect motor neurons against excitotoxicity and oxidative damage and stress. Preferred means of administering and amounts include the following:

For THC the effective dose range should be 10 mg-70 mg/kg/day intraperitoneally in rodents. Motor toxicity is seen at 84 mg/kg/day. An estimate of effective dose range in rodents can be obtained from comparison to other measures of bioactivity of these compounds, e.g. in analgesia and/or anticonvulsant activity. While maximum tolerated dose in humans must be established for each compound, with THC 10 mg/day orally is well tolerated, and up to 20 mg/day is approved by the FDA (IND #66435). However, other routes of administration may include intravenous, inhaled or sub-lingual, formulations such as in a patch or a spray, whereby the doses would most likely be different (e.g. 100 mg/day in a patch) in both rodents and humans.

Thus, administration of cannabinoids to ALS/MND patients in accordance with the invention is useful to provide protection. The anandamide/cannabinoid receptor/acceptor system offers a potential cellular mechanism by which both anti-excitotoxic and anti-oxidant effects may be achieved simultaneously, for a more substantial therapeutic effect in ALS/MND patients.

Also contemplated in practice of the invention is a screening method to screen for compounds promoting normal motor function in ALS patients. The screening method involves (a) administering a compound that is an anandamide/cannabinoid receptor/acceptor agonist to a mammal having observable motor function, preferably wherein the mammal has one or more ALS or MND symptoms, and (b) evaluating one or more indicia of motor function in said mammal, wherein a compound that promotes normal motor function is identified.

Alternatively, the screening method can be used to identify a compound for promoting increased survival of ALS patients. In addition, indicia of excitotoxicity and oxidative damage can be used to evaluate the effects of administering the compound.

The anandamide/cannabinoid receptor/acceptor agonist can be identified by contacting an anandamide/cannabinoid receptor/acceptor with one or more candidate compounds under conditions wherein dronabinol promotes a predetermined signal, identifying a compound that increases the predetermined signal, and administering the compound. The anandamide/cannabinoid receptor can be, for example, CB1 cannabinoid receptor.

Practice of the different aspects of the invention preferably is by use of an anandamide/cannabinoid receptor/acceptor agonist. Suitable anandamide/cannabinoid receptor/acceptor agonists include delta-9-THC, natural THC analogs, synthetic THC analogs, anandamide, and anandamide analogs. For example, the natural THC analogs include cannabidiol, which possesses antioxidant effects and is a partial agonist/antagonist at the abn-CBD cannabinoid receptor. Cannabinol is another natural THC analog with actions at the CB2 cannabinoid receptor. Delta-6-3, 4-transTHC is almost twice as potent an agonist at the CB1 cannabinoid receptor as delta-9, but is a minor constituent in marijuana. 11-hydroxy-THC is a metabolite of THC, which is also more active than its parent. Propyl-THC occurs in varying proportions in different strains of marijuana; it has less agonist activity than THC. The alkyl chain of THC is important in mimicking the conformation of anandamide at the receptor. Synthetic THC analogs include synhexyl and HHC; the latter is about ten times stronger an agonist than THC. HU-210 retains activity at central (CB1), but the isomeric compound dexanabinol (HU-211) is inactive, though it retains glutamate cascade cytoprotective action. CP55,940 is another synthetic analog about ten times stronger than THC. THC is not an alkaloid. Nitrogen-containing (alkaloid) analogs to THC include WIN55,212-2, an aminoalkylindole CB1 agonist, and SR141716A, a synthetic CB1 antagonist. Although its structure is quite dissimilar in the free state, anandamide wraps itself into the receptor in such a way as to mimic the shape of THC. Anandamide is an agonist at central (CB1) receptors and peripheral (CB2) receptors as well as the abn-CBD receptor. 2-arachidonylglycerol also activates CB1 and CB2 receptors. Palmitoylethanolamide acts at a CB2-like receptor and is implicated in non-CB1 neuroprotective action against glutamate excitotoxicity. Oleamide does not bind CB1, but may inhibit anandamide decomposition by anandamide amidohydrolase, or fatty-acid amide hydrolase (FAAH; below). Oleamide has been isolated from human cerebrospinal fluid in patients deprived of sleep. Other structurally similar fatty-acid amides probably interact with anandamide in competition at receptors, uptake pumps and enzyme sites; these fatty-acid hormone systems complement the action of the prostaglandins, steroids and peptides. It is likely that increased anandamide synthesis following ingestion of fatty foods contributes to the feeling of satiety and somnolence associated with such a meal. Synthetic compounds capable of antagonizing FAAH contain functional groups reactive towards catalytically active serine and cysteine residues on the FAAH enzyme. These include reversible and irreversible enzyme inhibitors.

Suitable formulations in practicing the present invention may also include pharmaceutical preparations with various inert, non-toxic, pharmaceutically suitable auxiliaries and excipients, as well as other pharmaceutically active compounds.

### EXPERIMENTAL

### EXAMPLE 1

Δ⁹-THC Delays Disease Progression and Improves Survival

hSOD1^{G93A} mice (ALS mice) were administered Δ⁹-THC (5 and 10 mg/kg body weight) or vehicle beginning at 60 days of age, i.e., prior to onset of disease signs. The earliest clinical signs of disease observed were tremors and shaking of their limbs when mice were suspended briefly in the air by their tails. These signs were never seen in non-transgenic littermates, but were always seen in hSOD^{G93A} mice after 75 days. In a subsequent set of experiments, mice were administered 20 mg/kg Δ⁹-THC beginning on day 75 when tremors were first observed, i.e., after onset of disease signs. Mice were evaluated on a rotarod to follow disease progression. No overt behavioral changes were observed in the Δ⁹-THC treated animals at any of the doses given. Furthermore, no significant difference in weights was observed between the treatment groups.

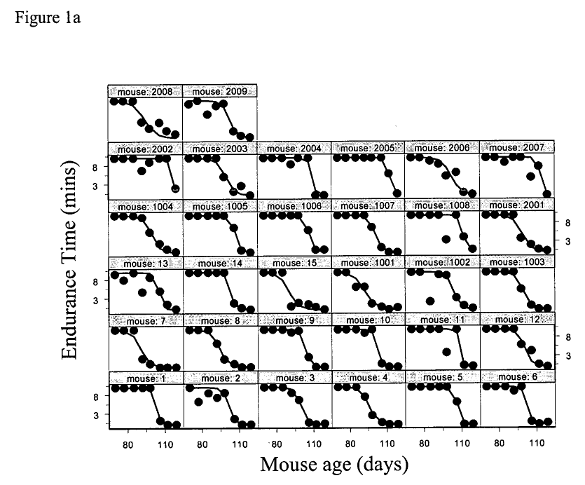

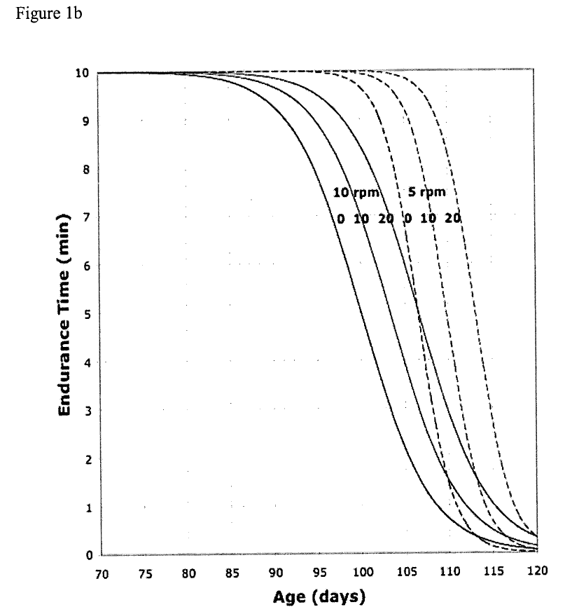

In order to assess the effect of Δ⁹-THC on disease progression, a logistic response curve was fit to the endurance time for each mouse using a nonlinear mixed effects model. For these experiments, each mouse was assessed on the rotarod at 5 and 10 rpm. FIG. 1A shows the observed data and fitted endurance curves at 10 rpm for each mouse. The results from the nonlinear mixed effects model showed that both dose and rpm (but not delay in treatment initiation) significantly affected age at which endurance declined to 50% (i.e. 5 minutes, abbreviated as A50% hereon). These results are summarized in Table 1 and shown graphically in FIG. 1B. A50% increased 3.3 days (±1.1 day) per 10 mg/kg of THC (2-sided p=0.003). In other words, disease progression as assessed by rotarod performance was delayed 3.3 days in the 10 mg/kg and 6.6 days in the 20 mg/kg group as compared to the vehicle treated animals. This represents a 3% increase in motor performance endurance in the 10 mg/kg group and a 6% increase in the 20 mg/kg group. Note that the increase in the 20 mg/kg group was not affected by the delay in initiation of treatment.

Animals were assessed later in the disease by testing them at the slower rotarod speed (5 rpm). In the data analysis, A50% increased 6.5 days (±0.4 day) when changing from 10 rpm to 5 rpm. However, the rate of decline increased by a factor of 2 in going from 10 rpm to 5 rpm. This rate of decline can also be understood by expressing it as number of days to decline from 9 minutes to 1-minute endurance, based on the fitting equation. At 10 rpm, it took 17.1 (±1.76) days to decline from 9 to 1 minute vs. 8.55 (±0.88) days at 5 rpm. These results are summarized in Table II and shown graphically in FIG. 1B. FIG. 1a. Δ⁹-THC delays progression of disease in hSOD1^{G93A} mice. Motor function was tested weekly on the rotarod at 10 and 5 rpm. The decline in endurance over time for each animal at 10 rpm is shown in (a). Mouse numbers 1-13 correspond to vehicle treated animals, numbers 1001-1008 correspond to the 10 mg/kg Δ⁹-THC treatment group, and 2001-2009 correspond to the 20 mg/kg Δ⁹-THC treatment group.

FIG. 1b Logistic response curves showing declines based on fitting a logistic model to observed data. Solid curves are tests at 10 rpm, dashed curves for 5 rpm. The three curves for each rpm are for doses of 0 (vehicle, blue), 10 (mg/kg Δ⁹-THC, green) and 20 (mg/kg Δ⁹-THC, red). Parameters for curves are based on a nonlinear mixed effects model given by Time=10/(1+exp((Age-A)/B)).

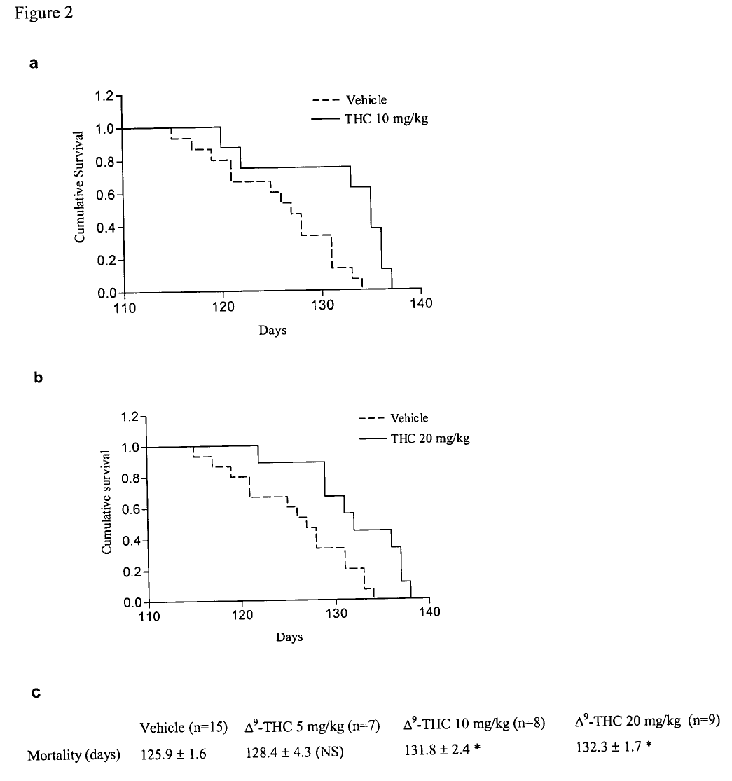

In these experiments, the treatment effect was to slow the progression of disease (FIG. 1b). Furthermore, treatment with Δ⁹-THC improved survival. There was a trend towards increased survival in mice treated with 5 mg/kg Δ⁹-THC (FIG. 2c). Treatment with 10 mg/ kg Δ⁹-THC extended mean survival from 125.9±1.6 days (vehicle, n=15) to 131.8±2.4 days (Δ⁹-THC, n=8, p<0.05)(FIG. 2a, c). This represents a 4.9 day (4.6%) increase in survival in the 10 mg/ kg Δ⁹-THC-treated group. At a (delayed) dose of 20 mg/kg the increase in mean survival was 6.4 days (5.1%).

FIG. 2. Δ⁹-THC extends survival in hSOD1^{G93A} mice. Cumulative survival in hSOD1^{G93A} mice treated with 10 mg/kg Δ⁹-THC (a) or 20 mg/kg Δ⁹-THC (b). Mortality of hSOD1^{G93A} mice treated with Δ⁹-THC or vehicle (c). Survival was significantly increased in the 10 and 20 mg/kg Δ⁹-THC treated groups compared to vehicle controls (*, p<0.05, error bars represent s.e.m.).

Δ⁹-THC is Anti-Oxidant and Anti-Excitotoxic in Vitro

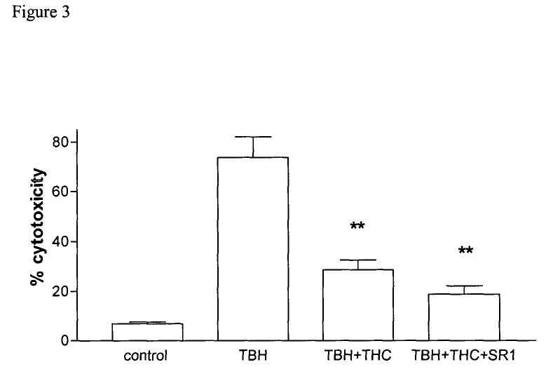

delta⁹-THC is known to be effective in attenuating in vitro excitotoxic and oxidative cell damage. Both of these mechanisms have been implicated in the progression of ALS. We had previously observed delta⁹-THC to be as effective as the anti-excitotoxic compound NBQX, an AMPA/Kainate receptor antagonist, in protecting spinal cord neurons against direct excitotoxin (kainate) exposure. To evaluate the possibility that Δ⁹-THC may also have antioxidant properties in spinal cord cultures, whether Δ⁹-THC could protect against oxidative damage produced by direct application of the oxidant tert-butyl hydroperoxide (TBH) was determined. Δ⁹-THC was extremely effective at reducing oxidative damage produced by TBH in mixed spinal cord cultures. Exposure to 200 μM TBH for 5 hours resulted in 74 (±14) % cytotoxicity, which was reduced to 28 (±8) % in the presence of 0.5 μM Δ⁹-THC (n=4, p<0.001)(FIG. 3). These quantitative data were visually confirmed by measuring propridium iodide uptake in parallel cultures (data not shown); both neurons and glia were affected by TBH as previously published. However, this profound antioxidant effect was not blocked by the CB, receptor antagonist, SR141716A (FIG. 3), suggesting the antioxidant effect was not CB, receptor mediated.

FIG. 3. Δ⁹-THC attenuates oxidative stress in mouse spinal cord cultures. Mouse primary spinal cord cultures were exposed to the oxidant tert-butyl hydroxide in vehicle (TBH), or in the presence of 0.5 μM Δ⁹-THC (TBH+THC) or 0.5 μM Δ⁹-THC plus 1 μM SR141716A (TBH+THC+SR1). Δ⁹-THC was extremely effective at reducing TBH cytotoxicity as assessed by LDH release; this effect was not reversed by the CB, receptor antagonist SR141716A (**, p<0.001, n=3-6, error bars represent s.e.m.).

The data presented here indicate that Δ⁹-THC delays progression of disease and increases survival time in hSOD^{G93A} mice even when administered after onset of signs. Our finding that Δ⁹-THC is both anti-excitotoxic and antioxidant in vitro suggests cellular mechanisms that may act additively towards its therapeutic effect in the hSOD^{G93A} mice.

TABLE I
Summary of Results

| Factor | Estimate | Std Error | DF | t-statistic | p-value |
|---|---|---|---|---|---|
| A. (Intercept) | 109.78 | 2.31 | 477 | 47.44 | <.0001 |
| A. (dose) | 3.29 | 1.12 | 477 | 2.95 | 0.0034 |
| A. (rpm) | −6.54 | 0.44 | 477 | −14.90 | <.0001 |
| B. (rpm) | 1.95 | 0.20 | 477 | 9.67 | <.0001 |

TABLE II
Predictions based on Model

| | | Days to 50% | Days from 9 to |
|---|---|---|---|
| rpm | dose | endurance | 1 min |
| 5 | 0 | 106.5 | 8.55 |
| 5 | 10 | 109.8 | 8.55 |
| 5 | 20 | 113.1 | 8.55 |
| 10 | 0 | 100.0 | 17.10 |
| 10 | 10 | 103.3 | 17.10 |
| 10 | 10 | 106.6 | 17.10 |

### EXAMPLE 2

This study was to determine maximum tolerated dose of dronabinol in ALS patients, as well as to gather data regarding disease course and symptom management. It is well established that glutamate levels in blood and CSF are elevated in ALS patients. The endogenous anandamide/cannabinoid (CB) receptor system effectively modulates glutamatergic neurotransmission and excitotoxicity. Anandamide/CB receptors in brain areas are associated with motor control, including their presence in mouse motor neurons.

Methods:

This was an open label crossover 7-month escalating dose study (from 2.5-10 mg) of Marinol treatment in 20 patients with ALS. The patients orally took tablets of dronabinol (Marinol®) for the treatment. Ten patients were randomly assigned to 3 months of Marinol treatment, followed by 3 months of no treatment, and 10 patients assigned to 3 months of no treatment, followed by 1-month washout and then 3 months of treatment. Each treatment arm was followed by a 1-month washout period prior to crossover to the next treatment arm. Patients were evaluated at baseline and monthly thereafter using Functional Rating Scale (ALSFRS) and Forced Vital Capacity (FVC), as well as the quality of life measure and analog scales measuring muscle fasciculations, spasms, appetite and sleep

Results:

Patients who were treated with Marinol in the first 3 months of the study were compared with those treated in the last 3 months. Primary efficacy variable of FVC and ALSFRS are compared between groups, as well as secondary variables evaluated. Marinol was well tolerated. Of the 25 patients treated with Marinol, one died in the first day after enrollment unrelated to drug effect. There were no other serious adverse events. Symptomatic benefits were seen in insomnia, appetite, fasciculations and spasticity while taking Marinol. Remarkably, 11 of the 19 patients showed reduction in rate of decline of ALSFRS while taking Marinol during a 3 month trial period. The average rate of decline was 27% when patients were taking drug.

These results show that dronabinol will reduce symptoms associated with ALS and possibly also slow disease progression.

It is to be understood that while the invention has been described above in conjunction with preferred specific embodiments, the description and examples are intended to illustrate and not limit the scope of the invention, which is defined by the scope of the appended claims.

It is claimed:

1. A screening method, useful to identify compounds affecting motor function in ALS or MND patients, comprising:
administering an anandamide/cannabinoid receptor/acceptor agonist to a mammal having at least one detectable motor function related to an ALS or MND symptom;
detecting any motor function change ; and,
evaluating any such motor function change with respect to a determinable motor function.

2. A method of screening for compounds useful for promoting normal motor function in ALS patients comprising: (a) administering a compound that is a anandamide/cannabinoid receptor/acceptor agonist to a mammal having observable motor function, and (b) evaluating one or more indicia of motor function in said mammal, wherein a compound that promotes normal motor function is identified.

3. A therapeutic method useful for a patient determined to be suffering from one or more abnormal motor symptoms of Amyotrophic Lateral Sclerosis or Motor Neurone Disease, comprising;
administering a predetermined amount of an anandamide/cannabinoid receptor/acceptor agonist, the amount administered being effective to promote normal motor function to the patient.

4. The therapeutic method as in claim 3 wherein the administering is oral, intravenous, inhaled, sub-lingual, or via a patch or a spray,
